(19) United States
(12) Patent Application Publication
(10) Pub. No.: US 2022/0023035 A1
(43) Pub. Date: Jan. 27, 2022

(54) TETHER LOCKING ASSEMBLY FOR AN ENDOVASULAR CARDIAC DEVICE

(71) Applicant: OPUS MEDICAL THERAPIES, LLC, Atlanta, GA (US)

(72) Inventors: Vivek Rajagopal, Atlanta, GA (US); Jaime Eduardo Sarabia, Mableton, GA (US); Yenchin Liao, Cary, NC (US)

(21) Appl. No.: 17/380,215

(22) Filed: Jul. 20, 2021

Related U.S. Application Data
(60) Provisional application No. 63/056,123, filed on Jul. 24, 2020.

(51) Int. Cl. A61F 2/24

(52) U.S. Cl. CPC A61F 2/2409; A61F 2002/0081; A61F 2220/0075; A61F 2230/0093

(57) ABSTRACT

A tether locking assembly for implanted cardiac medical devices, such as an artificial heart valve to replace the native heart valve, and which are secured at the implantation site by anchoring tethers secured to an intracardiac member, such as an intracardiac wall. The tether locking assembly includes at least one atrial positioning rod, at least one conduit, and a detachable lock. The detachable lock includes first and second hypotubes and a locking clip. The hypotubes are positioned within the positioning rod central lumen.

### CROSS REFERENCE TO RELATED APPLICATIONS

The application claims priority to U.S. Provisional Patent Application No. 63/056,123 filed Jul. 24, 2020, the disclosure of which is hereby incorporated by reference.

### FIELD OF THE INVENTION

The present invention relates generally to a tether locking assembly for implanted cardiac medical devices, such as an artificial heart valve to replace the native heart valve, and which are secured at the implantation site by anchoring tethers secured to an intracardiac member, such as an intracardiac wall.

### BACKGROUND OF THE INVENTION

Transcatheter valves have proven safe and effective for the replacement of native cardiac valves. These valves have been tested extensively for replacement of aortic, and pulmonic valves, but replacement of mitral and tricuspid valves remains challenging given the complex and delicate anatomy to which prostheses must attach. Anchoring transcatheter valves, in general, or transcatheter mitral and tricuspid valves, in particular, remains difficult, because doing so, either in the in-situ position of cardiac valves, or in other body lumens, requires interaction with a great variety of shapes and sizes of either cardiac valve annuli or other lumens. In this regard, the ability to tether transcatheter valves with one or more anchors affixed to intracardiac wall(s) provides greater security and flexibility for the transcatheter prosthesis by lessening need for fixation at the exact site of the prosthesis. For example, Applicants' Ser. No. 15/943,971 discloses an exemplary Transcatheter Anchor and Tether Devices, Systems and Methods of Implantation including an anchor delivery system for introducing a tether coupled to the anchor and a valve delivery system for delivering, positioning and sealing the valve.

### SUMMARY OF THE INVENTION

Presented herein is a detachable tether locking assembly for an endovascularly implanted medical device wherein the tether locking assembly is likewise implanted and locked utilizing endovascular techniques. For the sake of discussion, the tether locking assembly is described and shown used in connection with an atrial sealing skirt for a heart valve which is anchored to an intracardiac wall with tethers which extend from and anchor to the valve. The sealing skirt is positioned so as to be sealed at the atrial floor and secured in place by the detachable tether locking assembly.

The tether locking assembly comprises at least one atrial positioning rod, at least one conduit, and a detachable lock. The positioning rod has a proximal end configured to cooperate with an endovascular delivery system, and a distal end reversibly coupled to the detachable lock. The inner lumen of the positioning rod is configured for receipt of the tether wherein the positioning rod pushes or pulls the atrial skirt, thereby applying differential force and flexion to the associated top brim, allowing apposition to the atrial floor and/or conformation around an intracardiac lead. In another aspect, rotation of the positioning rod and/or pushing or pulling of internal elements of the positioning rod causes the detachable lock to engage the tether, or cord and/or suture connected to the tether, securing the cord, suture or tether to the valve (for example, atrial skirt), maintaining the force and flexion of the atrial skirt to atrial floor and/or intracardiac lead.

As shown and described, the sealing skirt includes a frame having a cylindrical shape, with the bottom of the cylinder at or below the valve annular level, and with the top of the cylinder extending into the atrium. According to one aspect of the present invention, the tether locking assembly is integrated with the valve. The valve, adjacent to the top brim, extends longitudinally along the interior or exterior of skirt body, includes one or more conduits, which take the shape of a cylinder whose cross-section is any portion of a circle, ellipse, parabola, or hyperbola, or take the shape of a polyhedron with a flat base and top which assume the shape of a polygon with three or more sides. These conduits are constructed from the membrane covering the skirt, or may be made of, but not limited to, stainless steel, nitinol or other metal alloys. The one or more conduits are hollow and accommodate at least one tether (or chord connected thereto), and each conduit attaches to a detachable lock near the atrial surface of the skirt.

The tether locking assembly includes a conduit (integrated or non-integrated with the atrial sealing skirt body), a detachable lock comprising a plurality of hypotubes, and a positioning rod for activating the detachable lock. According to one aspect of the invention, the conduit is integrated with the sealing skirt. According to another aspect of the present invention, the tether locking system is not integrated with the valve and the conduit is positioned adjacent the atrial side of the valve or medical device, the ventricular side of the valve or medical device, or remotely connected to the valve by the tether. The conduit shown is generally cylindrical with a cross-section in any portion is a circle, ellipse, parabola or hyperbola, or take the shape of a polygon with three or more sides. The integrated conduits are constructed from the membrane covering the sealing skirt or may be made of, but not limited to, stainless steel, nitinol or other metal alloys. The non-integrated conduits may be made of, but not limited to, stainless steel, nitinol or other metal alloys. The at least one conduit is hollow and accommodates at least one tether and each conduit cooperates with the detachable lock adjacent to or remote from the sealing skirt. The tether locking assembly includes at least one atrial positioning rod with proximal end attached to a delivery system, and a distal end reversibly coupled to the detachable lock, which, if the conduit is integrated with the sealing skirt, is attached to the proximal end of the conduit of the atrial skirt. Through the inner lumen of the positioning rod runs the tether wherein the positioning rod pushes or pulls the atrial skirt, thereby applying differential force and flexion to the associated top brim, allowing apposition to the atrial floor and/or conformation around an intracardiac lead. In another aspect, rotation of the positioning rod and/or pushing or pulling of internal elements of the positioning rod causes the detachable lock to engage the cord and/or suture, securing the cord and/or suture to the atrial skirt, maintaining the force and flexion of the atrial skirt to atrial floor and/or intracardiac lead.

The detachable lock includes a conduit, a first hypotube and a second hypotube and is positioned within the central lumen of the positioning rod. The second hypotube threadingly engages a locking clip positioned within a second hypotube and the first hypotube cooperate with tabs wherein removal of the first and second hypotubes secures the lock assembly position on the tether which, in turn, secures the sealing skirt position.

### BRIEF DESCRIPTION OF THE DRAWINGS

FIG. 1 is a side elevational view of the tether locking assembly having a conduit integrated with the sealing skirt and an atrial positioning rod connected to the detachable lock to lock the tether and secure the valve position;

FIGS. 2A and 2B are side elevational views of the detachable locking system of FIG. 1;

FIG. 3 is a side elevational view of the atrial sealing skirt illustrating the integrated lock conduit;

FIG. 4 is a top perspective view of the atrial sealing skirt illustrating the integrated lock conduit;

FIG. 5 is a side elevational view of the atrial sealing skirt having a non-integrated conduit positioned adjacent the proximal end of the valve and being operatively connected to the detachable locks and atrial positioning rods to lock the tethers and secure the valve position;

FIG. 6 is a side elevational view of the detachable locking system of FIG. 5;

FIG. 7 is a side elevational view of the locking assembly having a non-integrated conduit positioned proximally above the sealing skirt;

FIG. 8 is a side elevational view of the detachable lock of FIG. 7;

FIG. 9 is a side elevational view of the locking assembly having a non-integrated conduit positioned distally below the sealing skirt and adjacent the valve body;

FIG. 10 is a side elevational view of the detachable lock of FIG. 9;

FIG. 11 is a side elevational view of the locking assembly having a non-integrated conduit positioned distally below the valve;

FIG. 12A is a perspective view of the detachable lock prior to engagement with the tether with the positioning rod removed;

FIG. 12B is a cross-sectional view of the detachable lock of FIG. 12A;

FIG. 12C is a cross-sectional view of the detachable lock with the second hypotube retracted and withdrawn;

FIG. 12D is a cross-sectional view of the detachable lock with the first hypotube retracted and withdrawn;

FIG. 12E is a perspective view of the detachable lock engaged and locked with the tether;

FIG. 13A is a cross-sectional side view of the lock according to one aspect of the present invention;

FIG. 13B is a cross-sectional side view of the lock according to another aspect of the present invention;

FIG. 14 is a cut-away perspective view of a heart showing a transcatheter atrial valve positioned across the tricuspid valve in the heart;

FIG. 15 is a cut-away perspective view of a heart showing the transcatheter valve positioned across the mitral valve in the heart;

FIG. 16A is a perspective view of the atrial sealing skirt being positioned onto the right atrial floor by the atrial positioning rods; and

FIG. 16B is a perspective view of the atrial sealing skirt locked into position in the tricuspid annulus by atrial locks with the positioning rods partially withdraw.

### DETAILED DESCRIPTION OF THE INVENTION

The present invention can be understood more readily by reference to the following detailed description, examples, and claims, and their previous and following description. Before the present system, devices, and/or methods are disclosed and described, it is to be understood that this invention is not limited to the specific systems, devices, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. Those skilled in the relevant art will recognize that many changes can be made to the aspects described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

As used herein, the singular forms “a,” “an” and “the” include plural referents unless the context clearly dictates otherwise. Thus, for example, reference to a “tether” includes aspects having two or more tethers unless the context clearly indicates otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not. As used herein “fluid” refers to any substance that is free to flow and include liquids, gases, and plasma. “Fluid communication” as used herein refers to any connection or relative positioning permitting substances to freely flow between the relevant components. As used herein “integrated” refers to being contained by or adjacent to the corresponding member. The directional term “proximal” refers to the atrial side of the heart valve, in the direction of the delivery system and “distal” refers to the ventricular side in the direction of the anchor.

For the purposes of describing and defining the present invention it is noted that the use of relative terms, such as “substantially”, “generally”, “approximately”, and the like, are utilized herein to represent an inherent degree of uncertainty that may be attributed to any quantitative comparison, value, measurement, or other representation. These terms are also utilized herein to represent the degree by which a quantitative representation may vary from a stated reference without resulting in a change in the basic function of the subject matter at issue.

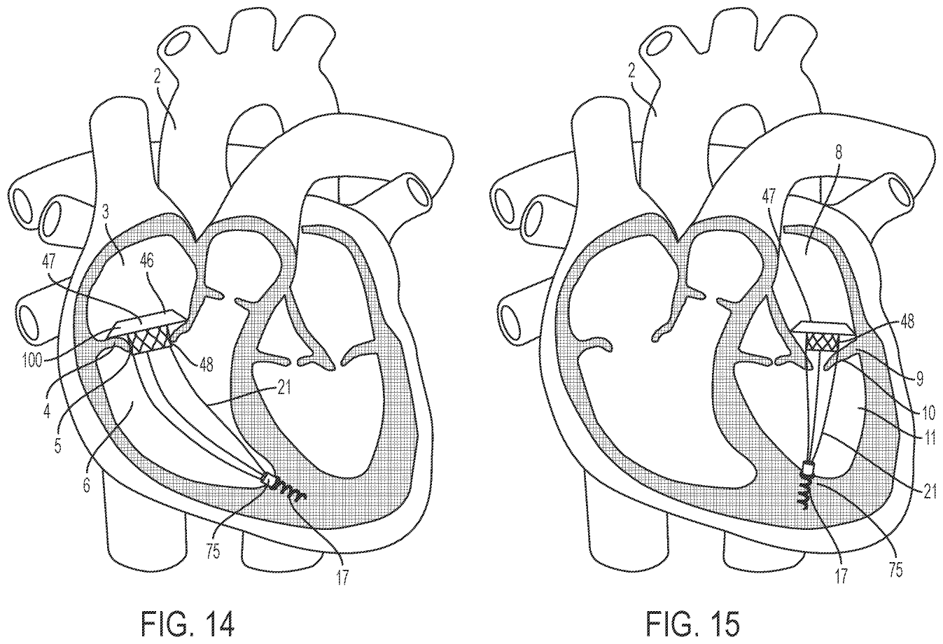

The detachable, tether locking assembly 10 as described herein is used in connection with medical devices and systems to be minimally invasively implanted in the heart to replace, to repair, or to support a native heart structure. By way of example only, the present invention as shown and described relates to a tether locking assembly 10 used with devices, methods and systems for endovascularly introducing and anchoring an anchor 75 (FIGS. 14 and 15) to the interventricular septum or intracardiac wall and implanting a valve 100 in the heart tethered to the anchor 75 to replace, repair or otherwise support the native valve. A tethering assembly including one or more tethers 21 cooperates with the anchor 75 connecting the valve 100 to the anchor 75. Furthermore, the valve assembly 100 includes valve body 48 and an atrial skirt top brim 47 cooperating with the valve assembly 100 to conform to the respective atrial floor to prevent paravalvular regurgitation of prosthesis.

As used herein, “tether” refers generally to a structure which extends from the anchor 75 secured to an atrial wall (interventricular septum or intracardiac wall) to the valve assembly 100 or another medical device employed. As disclosed in Applicant's prior application Ser. No. 15/943,971, “tether” refers generally to the cords of the tethering assembly comprised of the tether docking arm and chord or suture extending proximally therefrom. Alternatively, “tether” does not include a tether docking arm and is merely a tether in the form a tether cord extending directly or indirectly form the anchor 75 to the heart device, such as the valve 100. As used herein, the non-integrated conduit refers to a conduit which is not supported by the atrial skirt body 48. For example, the conduit's open upper end is not defined by the atrial skirt top brim 47 and is not fully supported longitudinally by the skirt body 48.

As shown, the heart valve assembly 100 includes a skirt body 48 and an atrial skirt top brim 47 extending circumferentially along the upper end of the valve 100. The skirt body 48 is generally cylindrical, and the atrial skirt top brim 47 is configured to conform to an atrial floor 4, such as the right atrial floor as shown. The atrial sealing skirt 46 is coupled to the anchor 75 by the tether 21 as described herein.

The transcatheter atrial sealing skirt 46 is sized and configured to sit in the tricuspid valve (in the example shown) between the right atrium 3 and the right ventricle 6 as illustrated in FIG. 14. The sealing skirt 46 may be pre-assembled with valve leaflets as an integrated valve or the sealing skirt may be constructed without valve leaflets and serve as a docking system for a separate transcatheter. This is by way of example. Optionally, however, with slight variations, the valve is sized and configured to be positioned in the mitral annulus between the left atrium 8 and the left ventricle 11 as shown in FIG. 15. When used in connection with replacement heart valves or a repair of a heart valve, the tether locking assembly of the present invention is used for either the tricuspid or mitral valves and may be endovascularly placed by a venous structure including, but not limited to, either internal jugular vein, either subclavian vein, either subclavian vein or either femoral vein. To facilitate endovascular implantation, the atrial sealing skirt 46 is self-expanding (i.e. the skirt is compressible so that it fits through a catheter of system 1) and composed of nitinol, but may also contain elements made of, but not limited to, stainless steel, nitinol or other metal alloys.

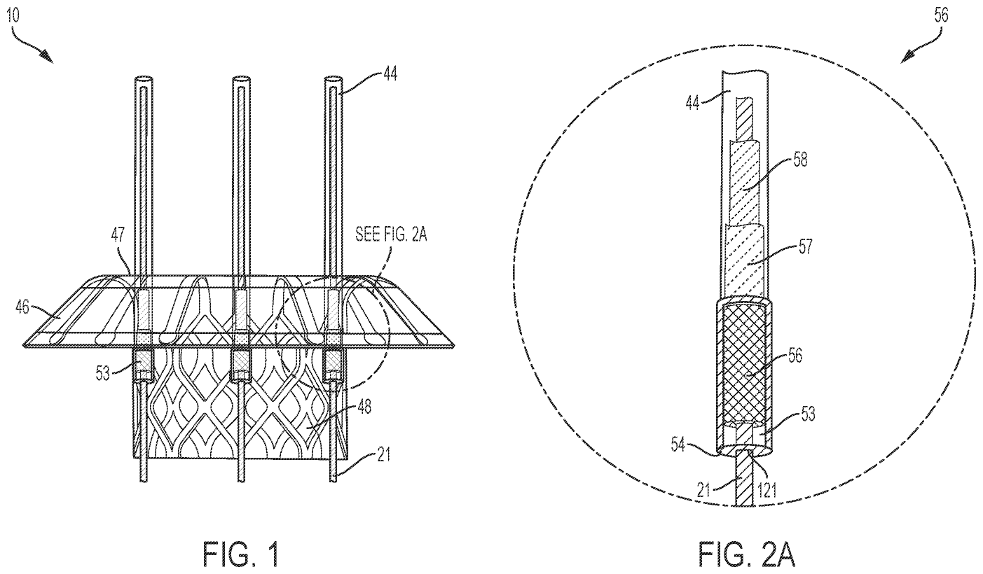

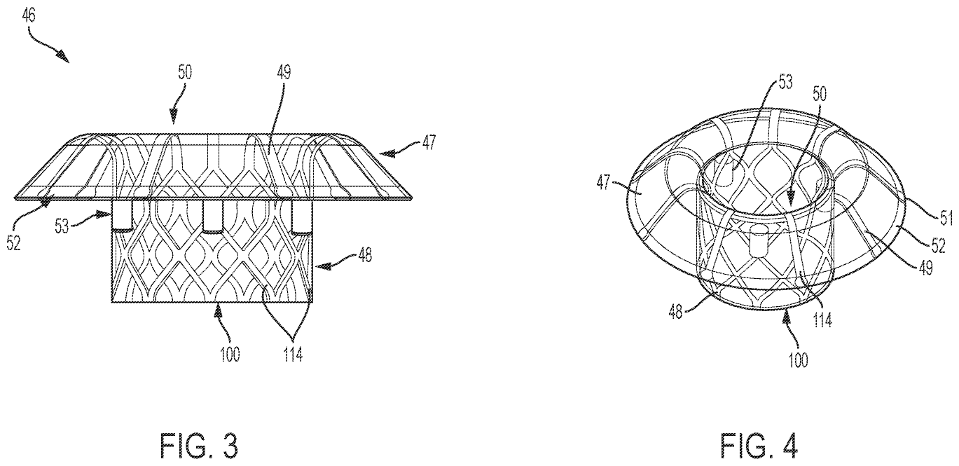

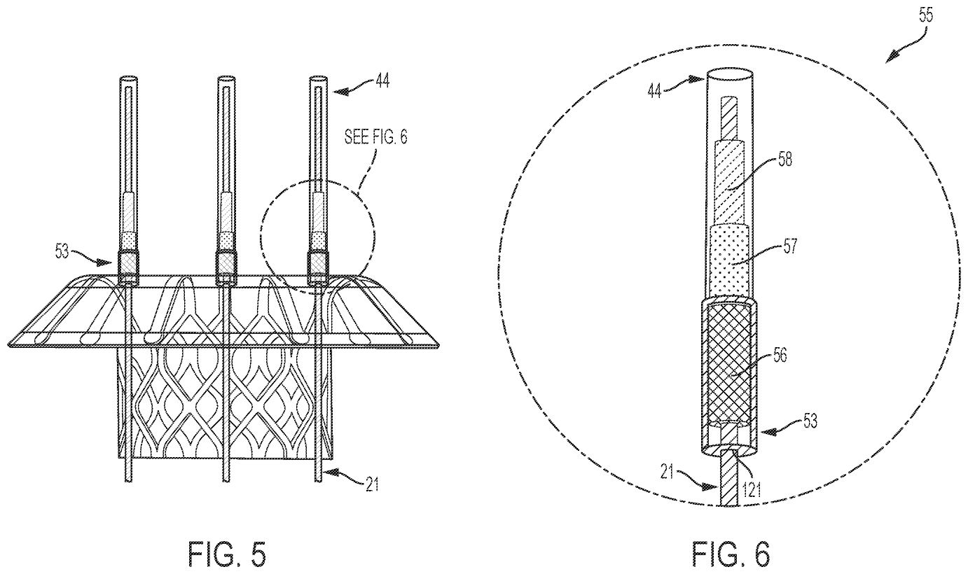

According to one aspect of the present invention shown in FIGS. 1-4, at least one conduit 53 is defined in the outer wall of the atrial sealing skirt 46. According to another aspect, shown in FIGS. 5-11, the conduit 53 is not integrated with the atrial sealing skirt 46. Each conduit is sized and shaped so that a portion of the tether 21 extends through the conduit aperture 121 and along the length of the conduit 53, thereby connecting the tether 21 to the atrial sealing skirt 46, allowing free movement until the skirt 46 is locked in place.

The at least one tether 21 is coupled, directly or indirectly to the anchor. In one aspect, the tether 21 may be a strong yet flexible cord such as, for example and without limitation, an expanded polytetrafluoroethylene (ePTFE) or ultra-high-molecular-weight polyethylene (UHMWPE, UHMW) cord. In use, described more fully below, a central portion of the cord 21 (between the distal end and the proximal end) extends through and/or be coupled to the atrial sealing skirt 46 to hold the skirt in the desired position relative to the tricuspid annulus or the mitral annulus.

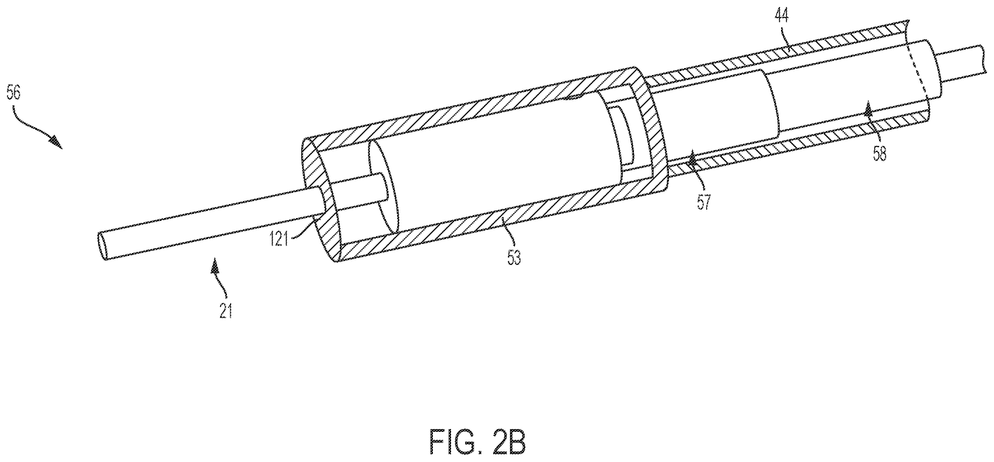

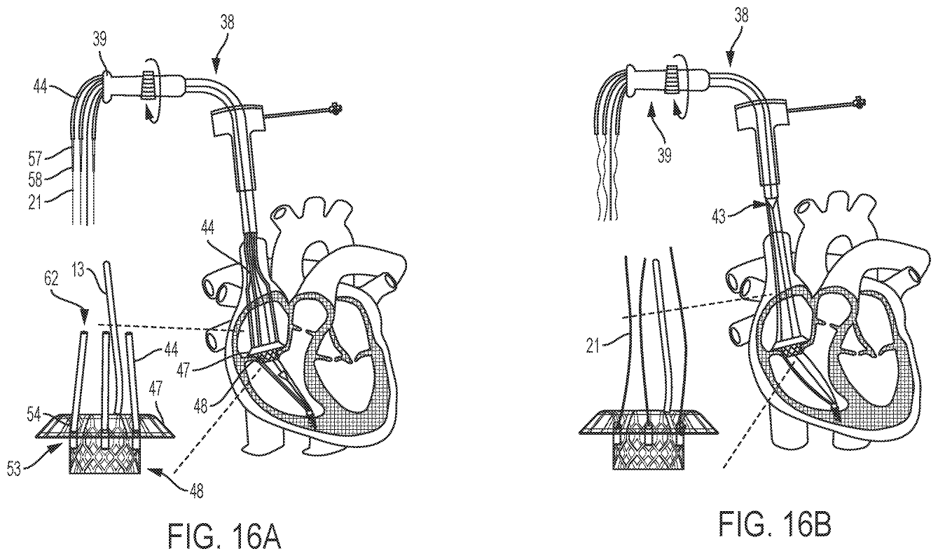

The tether locking assembly 10 includes a positioning rod 44 with a distal end 54, a proximal end 61 and an inner rod lumen 62 extending there between, the inner rod lumen is sized and configured so that a portion of the tether 21 extends therein. At least a portion of the atrial positioning rod 44 is flexible so that the distal end 54 of the atrial positioning rod may be positioned at or adjacent to the deployment site 5. The proximal portion of the positioning rod 44 extends beyond the delivery system, such as the delivery guide 38, as shown in FIGS. 16A and 16B. When shown in the various Figures, the length of the positioning rod has been shortened for the sake of discussion. For example, FIG. 2A depicts a break line and other Figures show a straight edge. It is to be understood that the straight lines depicting the proximal ends of the positioning rod 44, and hypotubes 57,58 are also not shown having an abbreviated length for the sake of discussion. As shown in FIG. 16A, the positioning rod 44 and hypotubes 57, 58 extend proximally beyond the delivery guide 38 and/or delivery knob 39.

The at least one positioning rod 44 is configured to couple with the conduit 53, when the conduit 53 is integrated with the valve as shown in FIG. 1, or when it is non-integrated such as in FIGS. 5, 7 and 9. The conduit 53 is shown as generally cylindrical, but other shapes are envisioned. The conduit 53 is sized and configured for receipt of the detachable lock assembly 10 and defines a distal aperture 121 configured to receive the tether 21 and having tolerances to permit movement of the tether 21 within the conduit 53. Referring to FIGS. 2A, 2B, 6, 8 and 10 the locking assembly 10 consists of a conduit 53 (integrated with the valve assembly 100 or non-integrated with the valve 100) which defines an inner receptacle configured for receipt of a plurality of hypo tubes. The first gateway hypotube 57 is sized and configured so that at least a distal portion thereof is received by the conduit 53 and the second hypotube 58 is sized and configured such that at least a distal portion thereof is received by the first retracting hypotube 57.

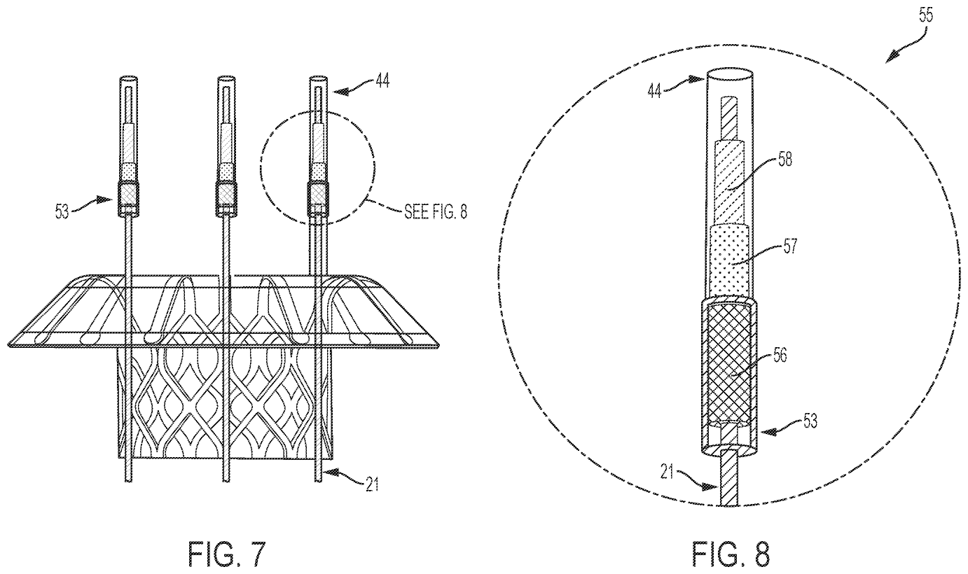

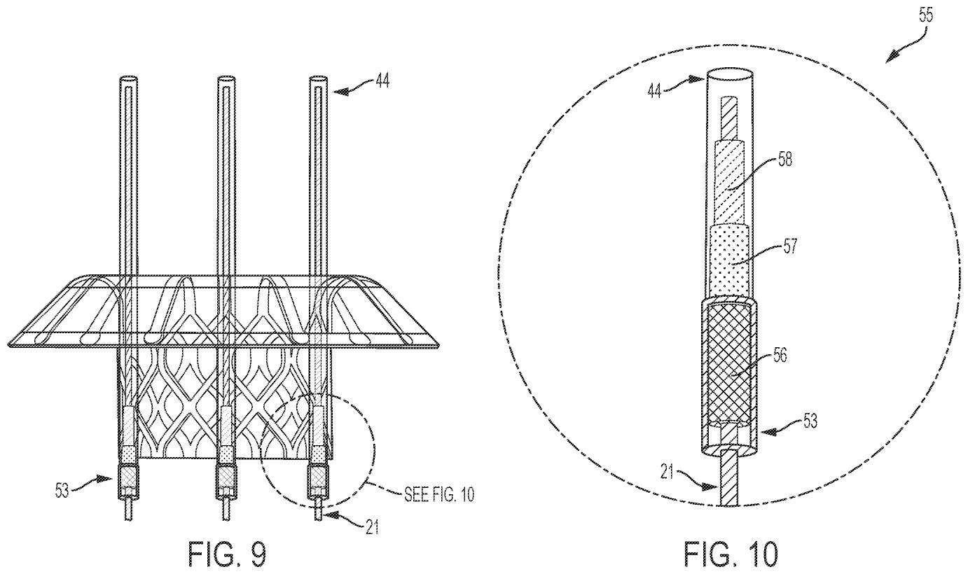

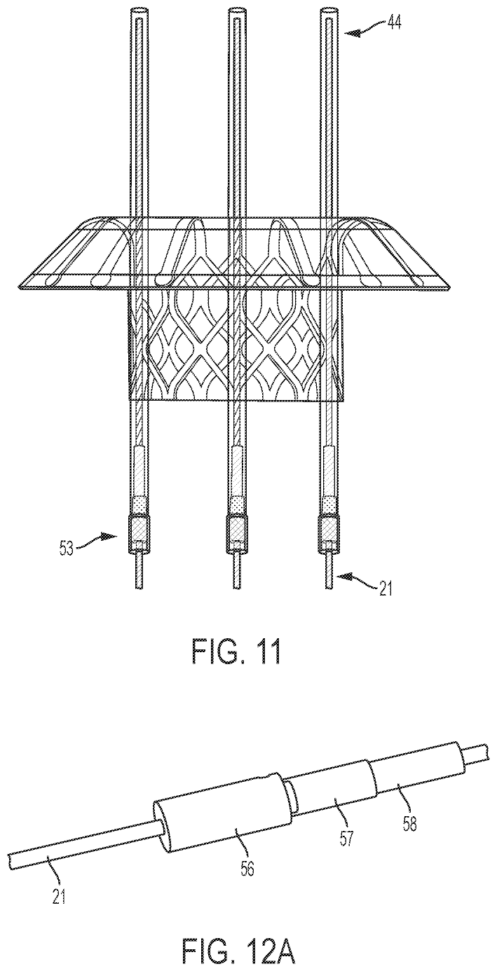

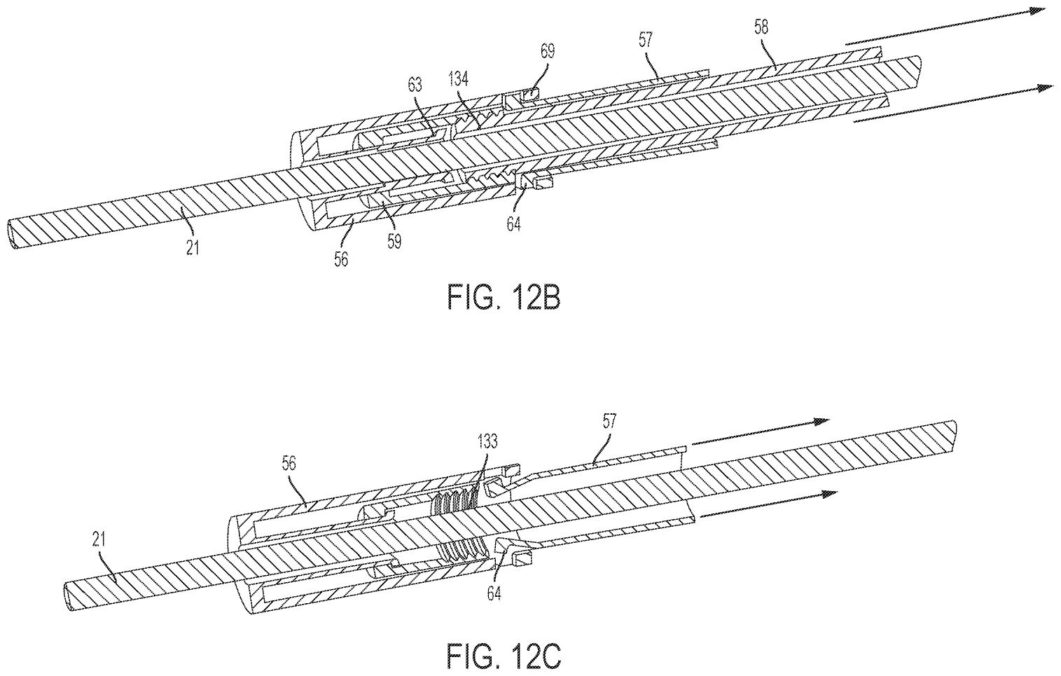

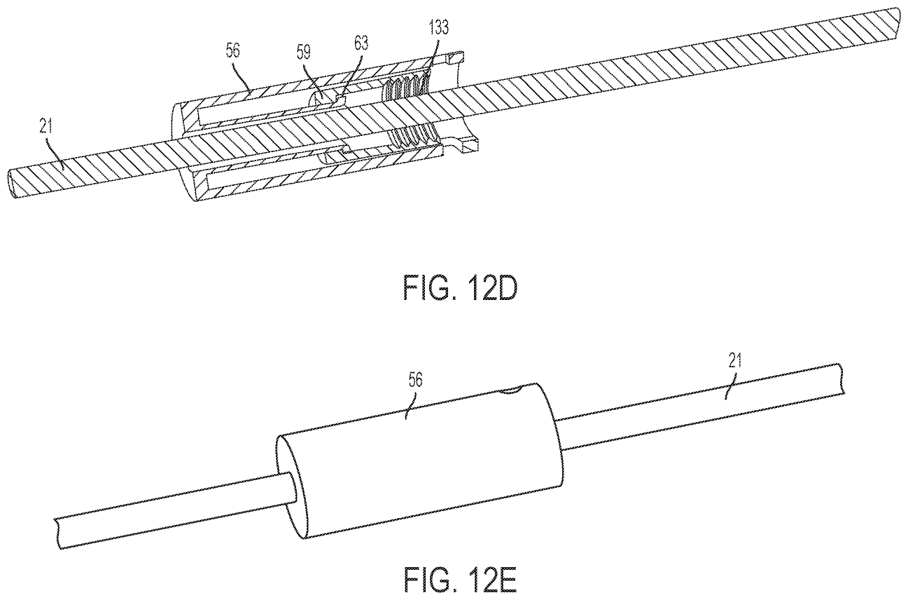

Inside the detachable lock 56 is a locking clip 59 shown in FIGS. 12B, 12 C, 12 D, 13A and 13B. The positioning rod 44, as shown in FIGS. 2A and 2B, is sized and configured to receive the detachable lock 56 including the first hypotube 57, the second hypotube 58. The distal end of the positioning rod 44, shown in FIG. 16A (lower enlarged image) contacts the sealing skirt to position the sealing skirt against the atrial floor. Thus, if the conduit 53 is integrated with the sealing skirt, the conduit is adjacent the distal end of the positioning rod 44 upon insertion and the two may be connected together. If the conduit 53 is non-integrated with the sealing skirt, but on the atrial side of the sealing skirt as shown in FIG. 7, the first hypotube 57 is positioned above the distal end of the positioning rod 44. And if the detachable lock is non-integrated with the sealing skirt and is positioned on the ventricular side of the sealing skirt, as shown in FIGS. 9 and 11, the first hypotube is positioned adjacent or distal to the positioning rod 44 distal end. The tethers 21 are sized and configured to be received by and cooperate with the conduit 53.

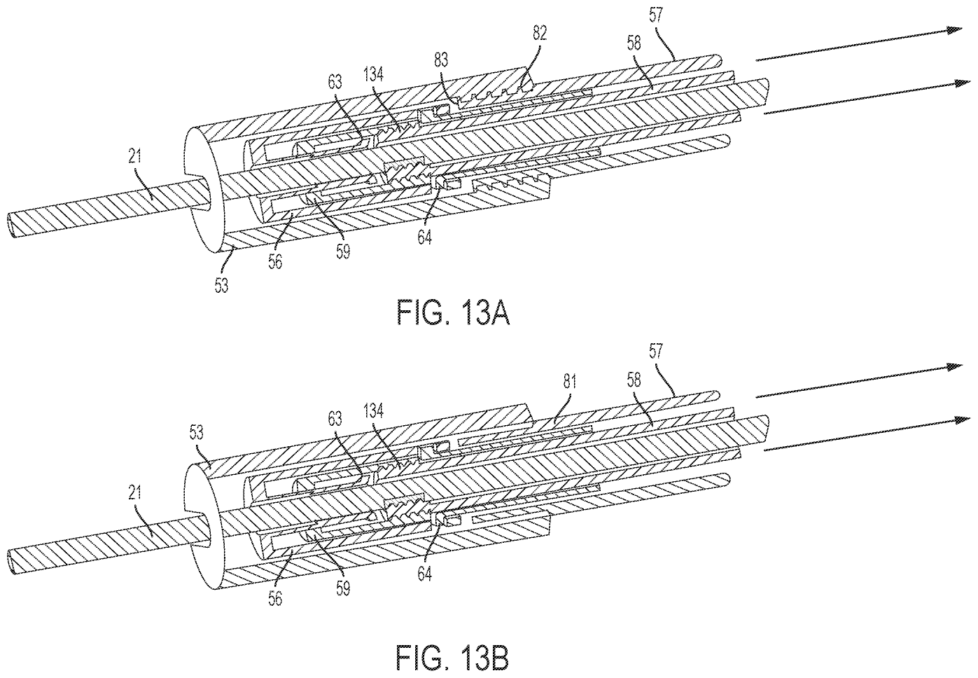

As shown in FIG. 13A, an inner surface of the lock conduit 53 defines a first mating surface 82 which is shown as a threaded area. A distal end of the positioning rod 44 defines a cooperating second mating surface 83, such as threads, to engage with the conduit first mating surface 82. Thus, the positioning rod 44 is threadingly disengaged from the conduit 53 by application of rotational forces on its proximal end.

According to one aspect of the present invention, as shown in FIG. 13B, the positioning rod 44 is integrated with the first hypotube 57. Accordingly, in use, the positioning rod 44 is removed (through the proximal end of the delivery knob 39 shown in FIG. 16A) and the first hypotube 57 is also removed in the same removal step. According to another aspect of the present invention, as shown in FIG. 13B, the positioning rod 44 and the first hypotube 57 are connected at a coupling 81 or may be formed as a single unit wherein a distal portion of the positioning rod 44 defines the first hypotube.

As shown in FIGS. 12B and 12C, (utilizing the positioning rod of FIG. 13A or 13B), the distal end of the second hypotube 58 defines a second mating surface 134 configured to cooperate with the first mating surface 133. Accordingly, the second retracting hypotube 58 may be rotated so as to disengage the cooperating threaded areas 133 and 134 to release the hypotube for removal by being withdrawn/retracted through the positioning rod 44 proximal end (the second hypotube 58 extends beyond the proximal end 61 of the positioning rod 44).

As the second hypotube 58 is retracted according to FIG. 12C or 13B, the clip 59 abuts locking tabs 63, thus pinching the tether 21. The second hypotube 58 is then released by unscrewing and according to FIG. 13A or the second hypotube 58 is retracted with the positioning rod 44 according to FIG. 13B. When the second hypotube 58 clears (i.e., is withdrawn past) the tabs 64 of the first hypotube 57 as shown in FIGS. 12B and 12C, the tabs 64 are biased inwardly and disengage from the locking clip flange 69 to release the first hypotube 57 from the locking clip 59 to enable it to also be retracted. When the first hypotube 57 is removed, the locking tabs 63 secure the tether 21 by continued radial, inward forces. FIG. 12D illustrates the lock 56 after the first 57 and second 58 hypotubes have been retracted. Thus, pulling of the retracting hypotube 58 causes retraction of locking clip 59, which pushes against locking tabs 63, engaging cord 21. Once the first hypotube 57 is removed, the clip 59 remains within the conduit 53 of the atrial sealing skirt 46. FIG. 12E shows a cut-away view of a lock fully engaged. FIG. 12D shows an intact view of a fully engaged lock.

The locking assembly 10 shown not integrated with the sealing skirt 46, FIGS. 5, 7, 9 and 11, are not positioned adjacent the distal end of the positioning rod 44, inherently, because the positioning rod 44 distal end cooperates with the sealing skirt top brim 47 to position the top brim 47 and secure it against the atrial floor (or around a lead). According to these aspects, the positioning rod 44 may or may not be secured to the hypotube 57. The positioning rod 44 may remain in place while the first and second hypotubes, 57, 58 are removed, or it may be removed prior to the locking assembly 10 becoming engaged.

In one aspect, the atrial sealing skirt 46 may be retrieved or repositioned. For example, if it is determined that the atrial sealing skirt is to be removed or repositioned, an atrial positioning rod 44 is positioned over each suture so that a portion of the suture is in the inner rod lumen 62. When the distal end 54 of the positioning rod is adjacent to or in contract with the detachable lock 56, advancing the first gateway hypotube 57 and the retracting hypotube 58 attaches the detachable lock to the distal end of the positioning rod, thereby unlocking the lock from the cord 21. With each cord unlocked, the valve may be removed from and/or repositioned in the deployment site 5.

In another aspect, the atrial sealing skirt 46 may be repositioned and/or removed days to weeks after valve deployment. In this aspect, the sutures are not cut, but wrapped around a spool or other wrapping device. This device is then attached to the valve on the atrial skirt top brim 47. Days after deployment of the valve and completion of the procedure, the spool/wrapping device may be re-captured, allowing un-wrapping and retrieval of the sutures. An atrial positioning rod 44 is then positioned over each suture so that a portion of the suture is in the inner rod lumen 62. When the distal end 54 of the positioning rod is adjacent to or in contract with the detachable lock 56, advancing the gateway hypotube 57 and the retracting hypotube 58 attaches the detachable lock to the distal end of the positioning rod, thereby unlocking the lock from the cord 21. With each cord unlocked, the valve is removed from and/or repositioned in the deployment site 5.

The tether locking assembly 10 is utilized after an atrial sealing skirt 46 (as shown) is implanted utilizing a transcatheter approach by placing a right or left ventricular anchor 75 and securing a tether 21 to the anchor 75. Once the valve is positioned at the implantation site, the tether 21, threaded and extending through the aperture 121 of the at least one conduit 53, is employed to secure the position of the valve. As shown in FIGS. 16A and 16B, at least a portion of the tether chord 21 extends along and proximally beyond the delivery guide such as the delivery rod such as the delivery knob 39 of the atrial sealing skirt delivery guide 38 shown. Thus, a portion of the at least one tether 21 extends through and beyond the distal end of the delivery guide 38, and a portion of the at least one tether 21 extends through and beyond the delivery guide 38. As shown in FIGS. 16A-16B, a proximal portion of the first hypotube 57 also extends proximally from the delivery knob 39 of the delivery guide 38 to provide access for retraction of the hypotube 57.

According to one aspect, the tether is pre-assembled with the valve 100 or the conduit 53 when it is not integrated with the valve, such that each tether 21 is threaded through the conduit. As such, the atrial sealing skirt 46 is moveable along the length of the at least one tether 21 until the desired deployment site 5 has been reached. That is, the atrial sealing skirt is free floating on the tether 21 until locked in placed by the detachable lock assembly 10.

When the atrial sealing skirt 46 is in the desired deployment site 5, the atrial positioning rod 44 is then be inserted over each tether 21 such that a portion of each tether 21 extends within inner rod lumen 62 and a portion extends beyond the proximal end 61 of the positioning rod 44. With reference to FIGS. 16A and 16B, the positioning rod 44 is then inserted through an atrial guide 38 and a portion of the tether 21 is received by the inner rod lumen 62 of the rod 44 and the distal end 54 of the positioning rod (with the detachable lock assembly 10 attached thereto) is adjacent to the conduit 53. The positioning rods 44 are pushed down by the user until the sealing skirt is in a desired position relative to the tricuspid annulus.

The atrial sealing skirt 46 position does not require pulling a tether 21 through the ventricular apex heart 2, because the atrial sealing skirt 46 moves freely over the tether 18 until the desired skirt 46 position is achieved. After the desired valve position is achieved, the at least one atrial positioning rod 44 urges the atrial sealing skirt 46 into position and is locked into place via the detachable lock assembly 10 within the conduit 53 and connected to the end of each positioning rod 44. The atrial sealing skirt 46 may be repositioned or retrieved until release of the tethers (or sutures extending therefrom) that extend through each atrial positioning rod 44

As shown in FIGS. 16A and 16B, the positioning of the atrial sealing skirt 46 inside the right atrium 3 so that the atrial skirt top brim 47 conforms to the topography of the right atrial floor 4 is shown. Via the atrial sealing skirt delivery system end 41, the practitioner advances one or more atrial positioning rods 44 so that the atrial sealing skirt 46 translates over one or more tethers 21, which extend through one or more conduits 53. As shown in FIG. 16B, as the atrial sealing skirt 46 advances toward the right ventricle 6, the atrial skirt top brim 47 contacts the atrial floor 4, and the one or more extension members 49 flex differentially according to the local anatomy. Because each atrial positioning rod 44 is pushed with differential force, precise tension amounts are achievable, and therefore more or less flexion of the extensions members 49 to facilitate conformation of the atrial skirt top brim 47 around the entire perimeter of the atrial floor 4 to limit regurgitation through the tricuspid valve orifice.

Although several aspects of the invention have been disclosed in the foregoing specification, it is understood by those skilled in the art that many modifications and other aspects of the invention will come to mind to which the invention pertains, having the benefit of the teaching presented in the foregoing description and associated drawings. It is thus understood that the invention is not limited to the specific aspects disclosed hereinabove, and that many modifications and other aspects are intended to be included within the scope of the appended claims. Moreover, although specific terms are employed herein, as well as in the claims that follow, they are used only in a generic and descriptive sense, and not for the purposes of limiting the described invention.

1. An atrial sealing skirt configured for receiving a valve and for endovascular introduction and implantation at a deployment site and configured and sized to replace a native heart valve, said atrial sealing skirt being configured to substantially conform to an atrial floor adjacent the atrial sealing skirt deployment site and comprising:
an atrial skirt body which is generally cylindrical, and which defines a valve receptacle;
an atrial skirt top brim extending circumferentially around an upper edge of said atrial skirt body, and wherein said atrial sealing skirt is compressible when constrained and expands when released from constraints;
a detachable lock defining a central lumen;
at least one tether extending through said detachable lock central lumen;
at least one conduit supported on said tether and defining a cavity configured for receipt of said detachable lock for securing said atrial sealing skirt on said at least one tether to lock said atrial skirt top brim against the atrial floor at the deployment site wherein said conduit is non-integrated with said atrial skirt top brim;
at least one extension member for supporting said skirt brim, said extension member having a base end adjacent said skirt body upper edge which extends outwardly substantially to an outer edge of said atrial skirt top brim;
at least one body support for supporting said skirt body; and
a membrane covering said at least one extension member and said at least one body support for forming the atrial skirt top brim and body.

2. The atrial sealing skirt according to claim 1 wherein said conduit is positioned adjacent a top surface of said atrial skirt top brim.

3. The atrial sealing skirt according to claim 1 wherein said conduit is positioned above a top surface of said atrial skirt top brim and is positioned a predetermined proximal distance from said atrial skirt top brim along said at least one tether.

4. The atrial sealing skirt according to claim 1 wherein said conduit is positioned below said atrial skirt body and is positioned a predetermined distal distance from said atrial skirt body along said at least one tether.

5. The atrial sealing skirt according to claim 1 wherein said conduit is positioned below said atrial skirt top brim and is positioned a predetermined distal distance from said atrial skirt top brim along said at least one tether and extending substantially parallel to said atrial skirt body.

6. The atrial sealing skirt according to claim 1 wherein said atrial sealing skirt defines a valve receptacle and comprises a valve within said valve receptacle.

7. The atrial sealing skirt according to claim 1 wherein said at least one extension member is non-linear.

8. The atrial sealing skirt according to claim 1 wherein said at least one extension member is a plurality of said extension members.

9. The atrial sealing skirt according to claim 1 wherein said at least one body support is a plurality of extension members forming a lattice-like structure.

10. The atrial sealing skirt according to claim 1 wherein said at least one conduit is at least two conduits.

11. The atrial sealing skirt according to claim 1 wherein said membrane is formed of a synthetic material.

12. The atrial sealing skirt according to claim 1 wherein said detachable lock is configured to be unlocked to permit repositioning of said atrial sealing skirt.

13. The atrial sealing skirt according to claim 1 wherein said detachable lock further comprises a first hypotube defining a central lumen and configured for receipt by said detachable lock lumen and configured to cooperate with said lock.

14. The atrial sealing skirt according to claim 12 further comprises a second hypotube defining a central lumen and configured for receipt by said first hypotube central lumen.

15. The atrial sealing skirt according to claim 12 wherein said detachable lock defines first cooperating surface and said first hypotube defines a second cooperating surface wherein said first and second cooperating surfaces cooperate to selectively retain said first hypotube within said detachable lock.

16. The atrial sealing skirt according to claim 1 wherein said atrial skirt top brim is generally concave prior to conformance with the atrial floor.

17. The atrial sealing skirt according to claim 1 wherein said conduit comprises a solid sidewall.

18. The atrial sealing skirt according to claim 1 wherein said membrane is formed of a biologic material.

19. The atrial sealing skirt according to claim 5 wherein said at least one conduit extends adjacent an outer side surface of said atrial skirt body.
